(19) United States
(12) Patent Application Publication
(10) Pub. No.: US 2026/0351361 A1
(43) Pub. Date: Nov. 5, 2026

(54) SECURE DIGITAL HEALTH DATA BANK

(71) Applicant: West Virginia University Board of Governors on Behalf of West Virginia University, Morgantown, WV (US)

(72) Inventors: Donald Adjeroh, Morgantown, WV (US); Neelofar Aleem, Cork (IE)

(21) Appl. No.: 19/194,560

(22) Filed: Apr. 30, 2025

(51) Int. Cl. G16H 10/60; G06F 21/31; G06F 21/60

(52) U.S. Cl. CPC G16H 10/60; G06F 21/31; G06F 21/602; G06F 2221/2103

(57) ABSTRACT

Various examples are provided related to secure digital health data banks. In one example, a method for secure data transfer to a digital health data bank includes login, via a user device, to an autonomous server of the digital health data bank, the login including an authentication based upon a sign-up QR code associated with a user of the autonomous server and a random question & answer (Q&A) challenge; generating, by the user device, a one-time QR code comprising user information encrypted based upon the sign-up QR code; and transmitting, by the user device, the one-time QR code to the autonomous server. The sign-up QR code is a unique identifier that is not directly linked to personally identifiable information of the user.

### BACKGROUND

The lack of centralized healthcare data and accessibility of a patient's record is a huge problem for clinicians as well as patients in the United States of America (USA). The lack of accessibility can have dire consequences. Patients who are not able to access their medical records, may end up undiagnosed with diseases such as circulatory diseases or cancer which is the major cause of death in the USA. This is despite the fact that 80 percent of all heart diseases are deemed preventable through proper in-time diagnosis that is only possible when patients have access to data when and as needed. There is a need for a secure system to save patient's medical history and records, tests reports, prescriptions, medical provider consultations and health data from IoT devices or apps, at one place.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of an autonomous dedicated server for secure digital health data bank access, in accordance with various embodiments of the present disclosure.

FIG. 2 illustrates an example of interconnected external systems with the autonomous health server, in accordance with various embodiments of the present disclosure.

FIG. 3 illustrates an example of a process of registration of users with an untraceable QR code, in accordance with various embodiments of the present disclosure.

FIG. 4 illustrates an example of a process of call and response authentication in accordance with various embodiments of the present disclosure.

FIG. 5 is a flow diagram illustrating an example of a methodology to create random Q&A challenges, in accordance with various embodiments of the present disclosure.

FIG. 6 illustrates an example of data transfer, device verification, and data processing and analysing, in accordance with various embodiments of the present disclosure.

FIG. 7 is a flow diagram illustrating an example of drawing of seamless health data gathering and storing, in accordance with various embodiments of the present disclosure.

FIG. 8 is a flow diagram illustrating an example of data processing and data recording for an AI simulation model, in accordance with various embodiments of the present disclosure.

FIG. 9 illustrates an example of a user specific digital health object and its use in healthcare, in accordance with various embodiments of the present disclosure.

FIG. 10 illustrates an example of a computing environment for secure digital health data bank access, in accordance with various embodiments of the present disclosure.

### DETAILED DESCRIPTION

Disclosed herein are various examples related to secure digital health data banks. Reference will now be made in detail to the description of the embodiments as illustrated in the drawings, wherein like reference numbers indicate like parts throughout the several views.

A “One Stop All” digital health platform is introduced to record all health data across multiple services, referred to as a Digital Health Data Bank (DHDB). The proposed system is a database of all essential patient healthcare information. This digital interface is based on the modern-day digital banking system and provides access to all health data digitally. The DHDB can help many fields. The DHDB provides valuable information on what medical devices will need in the future based on collected DHDB data about current disease trends. Using a single platform, all of the patients' medical records and healthcare history can be accessible and thus helps in diagnosing chronic illnesses at the right time with possible early diagnosis. It can help fight chronic illnesses and addressing critical health issues, as using the DHDB can lead to early research and planning for future pandemics. An advantage of the DHDB is that it can store all types of health or medical data associated with a patient. This technological solution connects multiple health services. Patients can store and retrieve information from connected health devices and multiple applications using a single interface. Clinicians can access the patient medical records with the patient's consent. Moreover, if the patient agrees to use their records for medical research (anonymously), it can immensely benefit in medical research breakthroughs and innovative development for the future. As the DHDB can access information from multiple sources, including health check-up and diagnosis at hospitals, lab test reports, prescriptions for pharmacies, health insurance claim details and IoT devices data, thus, the DHDB can connect various sectors of healthcare such as hospitals, labs, m-health service providers, or even health insurance companies using one platform. All these aforementioned functions and advantages can help evolve a central digital health care system in the USA and beyond.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principals disclosed by the following examples.

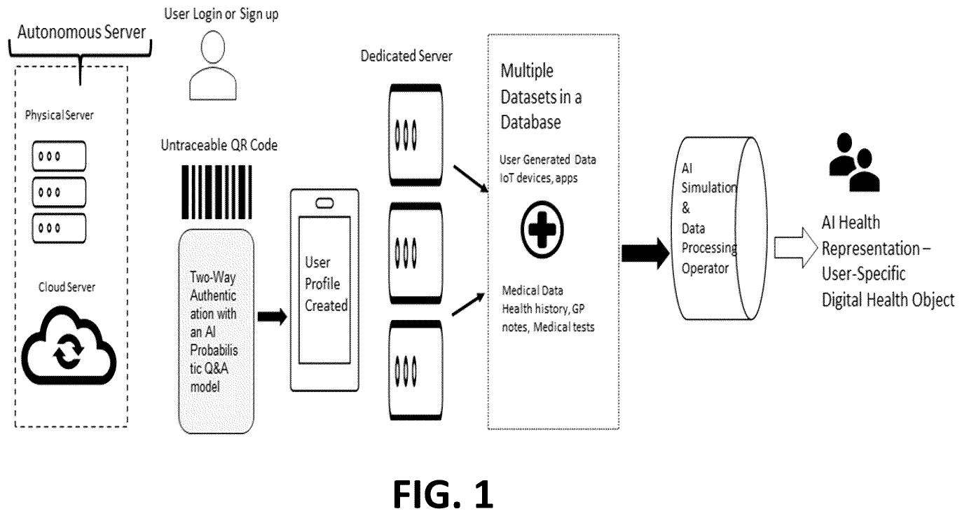

With reference to FIG. 1, shown is an example of the DHDB presenting an overview of various functionalities. Any process descriptions or blocks in flow charts should be understood as representing modules, segments, or portions of code which include one or more executable instructions for implementing specific logical functions or steps in the process, and alternate implementations are included within the scope of the preferred embodiment of the present disclosure in which functions may be executed out of order from that shown or discussed, including substantially concurrently or in reverse order, depending on the functionality involved, as would be understood by those reasonably skilled in the art of the present disclosure.

As illustrated in FIG. 1, an autonomous dedicated server can include physical server(s) and/or cloud server(s) designed for an individual user. To login or sign up, first an untraceable sign-up QR code is generated by the system and then the user answers a question from an AI probabilistic random Q&A to create a profile. Each user profile collects, stores and manages datasets from multiple sources which can then be transmitted to an operator for processing information. Based on this operator's analysis, the system can predict individual health trends.

This autonomous health server is a self-managing server that operates with minimal human intervention. The benefit of this autonomous health server is that it stores data from other devices, shares files, controls the system from another device or app, and enables multiple data devices combined on one single place. Encryption is used to provide a high level of security while using this server. Having this autonomous health server is like owning an autonomous vehicle, where the user does not need to actively work in order to have control over the system. A dedicated server assigned to a single user links with the system's main server to host and run the users' request and helps users to access all services. The main system server manages and maintains the physical server resources and allocates them to different users as needed. This server works for the user's healthcare. This autonomous health server can be created as follows.

The autonomous health server is based on a combination of physical servers and cloud servers. First a physical server is made using a hardware with memory, hard drive, network connectivity, and operating system. To combine two and more physical servers together, the system can use a direct cable with two patch cables connected one to each server. Then system generates IP addresses for each server available in the same physical range. This physical server machine is then connected with many virtual computers (with IP addresses) and co-exist with cloud databases. The code can be written to collect data from multiple devices and online sources and to automatically distribute data across all databases. A simple cloud server can be created (i.e., virtual not physical) server running in a cloud computing environment that can be accessed on demand by user. Cloud servers working alongside physical servers increases efficiency of the overall system. The server limits who can connect and allows devices on the same network and with user credentials to link with the server only. The server exchanges the data with the system server, encrypted using HTTPS to prevent eavesdropping. As the server is accessible over the internet, a firewall Network Address Translation (NAT) can block unauthorized connections unless specifically allowed by the user.

After combining cloud and physical servers at the system's end, when the system (as an application) is downloaded to a user's device, the user's device would act as a physical server connecting it with the cloud server of the main system to manage and maintain information coming from multiple sources to the health database. To create a security layer, the user is asked to authenticate (with the sign-up QR code and call and response probability Q&A) before the device acts as a server. In addition to this, the encryption can be done using HTTPs protocol to secure data transit. When an app sets up a server, it can use a networking protocol, first HTTP (for web communication) and then WebSocket (for real-time data). The user end server is configured to accept data only from trusted sources which are authorized by the user. This server opens a specific port on the user device to manage incoming requests from different health databases and responds to those requests. The server runs as part of the app process, so when the user closes the app, the server stops too. This saves battery of user's device and network usage; the server stops working when user closes the application.

The autonomous health server is created using both the user's device and the system server (cloud and physical servers). In python, flask framework can be installed to create a local server. The flask framework allows setup of endpoints and uploading health and images including health data in Java script Object Notation to format excel sheets in a readable way, allows user to upload images, automatically retrieves and decrypts health data for viewing (like in Health Language 7 or health app) and creates a metadata base. The libraries from flask python can also be added. The data can be collected from any or all of the following devices or applications: Health data from Apple Health by using HealthKit API, available only on iOS; (the system developed on Python won't directly access this data rather receive the exported data in other acceptable formats via the server) Samsung Health, (uses Android's Health Connect API to export data); Users uploading any image (MRI, X-rays) data using a web interface; Smart watch (using Android and other operating systems), collects data including user's exercise, sleep, heart rates, pulses and any other data and stores in xml/JSON format; From Electronic Health Records (EHR), data from hospitals, pharmacies and medical testing labs taking data in HL7 formats; or Data from other sources supported by the server and this system.

High-Level Encryption: Data can be encrypted both in transit (HTTPS) and at rest (AES encryption for storage). This system uses the cryptography library for AES-based encryption and Fernet cipher to handle encryption and decryption of sensitive data. The flask in python runs behind an HTTPS-enabled server to do encryption of in transit information and to ensure secure file handling uploaded files are saved in their respective dedicated databases—secure directories using secure filenames to prevent in-transit path traversal attacks. Also, sensitive data can be stored in an encrypted format using cipher suite encrypt.

Users can be allowed to export data via the Health app on their mobile devices and this exported data is in XML/JSON format, which can be uploaded to the server via the defined endpoint. The system accepts data exports manually as well and accesses it using Health Connect APIs over an established secure communication. The servers autonomously make decision on replacing in-memory storage (like securing recent health data first) with a secure database of MySQL, as this creates scalability. The libraries used for Android systems are Java and Nano HTTPD as a server library and Swift for iOS.

The server can interact with the device's network stack, allowing it to perform multiple functions. The server can be accessed only within the same device or local network and allows nearby devices to connect. It has proper configuration; thus, the server requires additional permissions and security measures for running on a users' device. The local server in user's device access files stored on the phone and interact with devices available on the users' Wi-Fi network. To set up a server, the app needs certain permissions: including network permissions to open a port and communicate and background permissions to run server while the app isn't active.

The autonomous health server facilitates automation of tasks such as configuration, monitoring, and maintenance. This autonomous health server provides a dedicated system to each user. The autonomous health server collects health information from many places similar to an autonomous vehicle that collects information from its'surroundings. This multi-model autonomous server receives data from multiple sources and stores it in a combined cloud and physical server system.

It can also help in creating an artificial intelligence (AI) agent for each user (which can help in medical diagnosis of disease and testing treatments on the digital health object before the actual person takes it). These AI agents can aid in making decisions and taking actions for diagnosis and testing treatments in digital environments with least human involvement. The usage of these agents is safe because a diagnosis is only written description of an AI agent that can be submitted to the user's doctors or health providers for further discussion and evaluation. By having more information about the user, these AI agents can act as digital health objects of the users and test treatments.

The servers are designed to receive data from multiple sources using a scalable architecture to ensure reliable and efficient data handling. Incoming data is directed through a load balancer to distribute data receiving requests across multiple servers to prevent any single server from overloading which relies on queues. The unique design of data processing software can use AI and Machine Learning (ML) models to process multiple sets of data at once. This system would receive and store diverse types of data about a user (patient), such as written data in words (e.g., doctor's notes, diagnosis, written prescriptions, etc.), medical scans (e.g., X-rays, MRI scans, images of biopsies, ultrasound, etc.), medical reports (e.g., blood test reports stating amount of iron in blood, etc.), IoT devices data (e.g., heart beat rate during sleep or workout equipment data, etc.), medical transactions (e.g., medical bills, insurance claims or bank payments, etc.)

For each of these data sets, diverse sets of information (reference values) are used to process, analyze and make decisions. For example, a blood test report showing hemoglobin levels of female ranges between 115-160 which is a numerical value versus analyzing an ultra-sound image (e.g., tracing bone, tissue or fluid) is visual effect processing. Thus, the software would be based on stored references to process and analyze multiple data sets at once and draw conclusions.

To resolve queuing, delayed buffers and data loss issues, this server can be based on dynamic buffers and queues that are resizable based on the amount of incoming data. It can manage high-frequency data streams, ensuring messages are processed sequentially and without data loss. To standardize diverse data formats, the data ingestion layer (using APIs, microservices, and streaming protocols) manages, validates, and transforms incoming information in real-time. The data receiving server send all this data to storage which involves databases optimized for both real-time and long-term data access, integrated with data lakes or warehouses for analytics. Each user connects to the system through a unique interface designed to accommodate the nature of their data, scale of input, and access permissions. All data is recorded in the local server storage and cloud-based system.

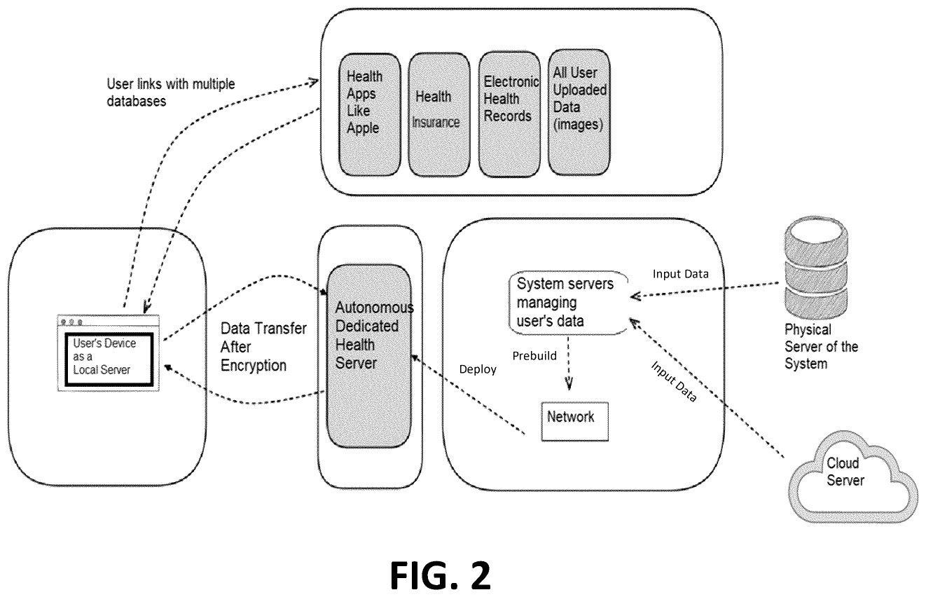

With reference to FIG. 2, shown is an example of interconnected external systems with the autonomous health server according to various embodiments. FIG. 2 draws on how the autonomous server system works. The server connects with the external databases for multiple services. It acts as a hub for collecting, processing, and distributing data from various sources and taking information from health apps, health insurance, electronic medical and health records, and uploaded data by the users. This architecture enables integration of data across different platforms, allowing server to harness the power of diverse datasets for enhanced decision-making, operational efficiency, and user engagement.

At the core, the autonomous health server serves as the primary hub for the collected data. The interconnectedness enables databases to perform comprehensive analyses across different touchpoints. The centralized system can communicate with other information sources to deliver relevant data to the users' server. This communication can involve using server-side scripting languages to ensure high level encryption. FIG. 2 illustrates the server, and the way the autonomous health server can retrieve the needed information and present it in a user-friendly format. The use of encryption allows this system to enhance security of healthcare data.

This system can be built on different coding languages including python as main coding and other sub languages (like PHP, HTML) to balance different functionalities. PHP handles dynamic web content generation and server-side logic, MySQL for database, Python focuses on AI-driven data analysis and complex processing and HTML structures the front end for the final display of data.

To ensure privacy of users, an unrecognizable sign-up QR code authentication is embedded for security. The app can utilize seamless data receiving based on multi-threading (a parallel thread for each user communication is created) to support the system's responsiveness for big data receiving and data protection. This sign-up QR code serves multiple purposes: it is designed to be untraceable, ensuring that the user's real identity remains anonymous. It acts as a unique identifier that is not directly linked to any personally identifiable information (PII). This ensures user privacy while still allowing the system to identify and authenticate users without relying on their real-world identity. This authentication process is dynamic in a way that user can enter any personal information they want (like name, location, etc.).

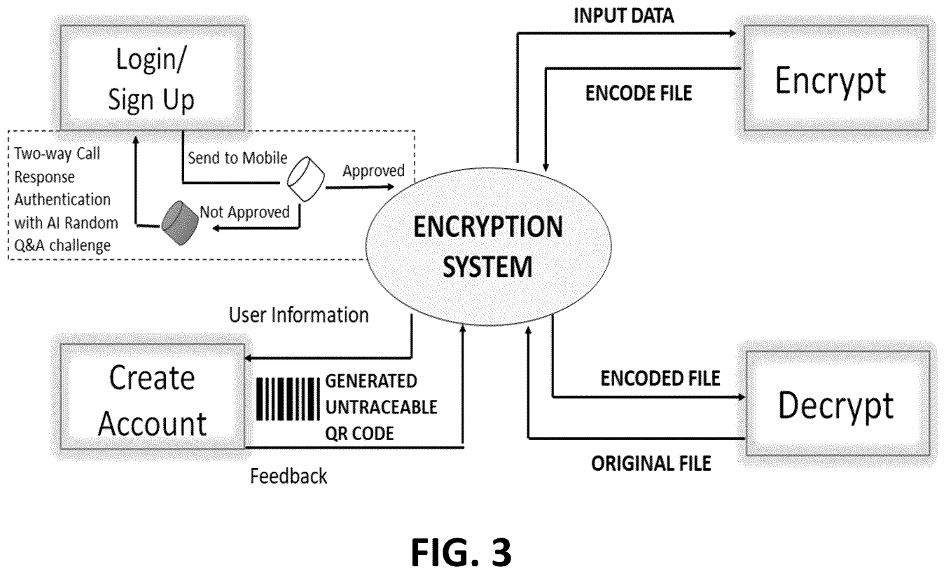

With reference to FIG. 3, shown is an example of a process for registration of users with an untraceable sign-up QR code. The system of FIG. 3 is designed to ensure the security and privacy of individuals' data during the login or signup process. An exemplary process for creating a user login is illustrated on the top left of FIG. 3. To enhance security, the system can use two-factor authentication, requiring the user to verify their identity in two steps. First, the user is required to enter the standard login credentials (username and password). These credentials are sent to the server using a secure HTTPS connection, protecting them from interception during transmission. Second, after entering the correct username and password, the user must verify their identity through a one-time passcode (OTP) sent as an SMS or email. The user must input this code to proceed. The user enters this code into the system to complete the login or signup process.

Once the user successfully logs in or signs up, the system immediately encrypts any sensitive data entered by the user. This central encryption system is responsible for protecting user's personal information, such as names, email addresses, phone numbers, or any other sensitive data.

As shown in the right side of FIG. 3, the encryption system supports a two-way flow of information, ensuring that data can be both encrypted and decrypted securely. When a user submits personal data (e.g., during the sign-up process), the system encrypts this data using the encryption keys. The encrypted data is then stored in the database or transmitted across networks. When the system needs to retrieve and use the encrypted data (e.g., displaying a user's profile or generating reports), the data is decrypted using the same encryption key that was used during encryption.

After successful encryption and processing of the user's data, the system employs a novel approach to account creation by generating a special, untraceable sign-up QR code for each individual user. This sign-up QR code serves multiple purposes: The sign-up QR code is designed to be untraceable, ensuring that the user's real identity remains anonymous. It acts as a unique identifier that is not directly linked to any personally identifiable information (PII). This ensures user privacy while still allowing the system to identify and authenticate users without relying on their real-world identity.

Next, a random one-time token is generated. This token is encrypted with the transaction details provided by the user at that time (using recipient's public key). Using the encrypted details and token, the one-time QR code is encoded. When the user scans the one-time QR code and sends the encrypted data to the system's server, the server resolves it to the intended action without storing personal information of the user.

Below is a detailed explanation of steps that can be followed to create untraceable QR codes for users of this system with a high level of privacy while ensuring measures for cyber security.

The QR codes can be encoded based on non-personal identifiers containing a system-generated identifier. When the user signs up, a unique sign-up QR code is created using random numbers with no inherent connection to the user. This cannot be reversed to reveal user details. Every time the user logins into the system a new one-time QR code is generated with the information that only remains valid for that single session or specific transaction and expires after the session. However, unlike other systems using one-time untraceable codes or keys, this system requires users to show the previous sign-up QR code created at the time of initial sign up. This ensures that even without username or email, users' need to go through the measures of cyber security and validity authorization.

When the sign-up QR code is generated at the users' sign up, it connects with the autonomous server. The server uses a combination of physical servers (local server of the system and the users' device) and cloud servers. Even though the sign-up QR code links with one user, multiple servers allow the system to route data in a decentralized way. Thus, one-time QR codes generated every time the user logs in are unique for each time and are not tied to the user details in a central location. These QR codes facilitate direct interaction with the users' database eliminating intermediaries storing data. Only the user can store information in a central location coming from many databases.

The sign-up QR code is used as a user profile (to store and process users' health data anonymously) and acts as the main encryption key for Recording health data anonymously and ensuring users' privacy. Other one-time QR codes are generated at the users' local server (users' system without IP or location information) and are only used to sign in as a single event and not to be reused. Thus, two types of QR codes are generated: first is the sign-up QR code and second is the one-time QR code. The data is encoded and linked to the sign-up QR code.

As shown in FIG. 2, the users' device (e.g., smartphone, tablet, laptop computer, or other computing device comprising processing circuitry with processor and memory and communications capabilities) would act as a server and hence each time the user logs into this system, a one-time QR code can be generated on users' device without connecting it to the outside server. Also, when the first-time users install this app system, the QR code studio software would be installed into users' device to generate QR codes without external server interference. This system can generate QR codes without any outside tools (like Python) and creates a QR codes library of its own where sign-up QR codes are stored. The system can use a custom bitmap generator programming environment that supports the graphic rendering which helps in a standard QR Code generator and detects errors. The custom bitmap generator programming environment can be created following these steps.

First, the data given by the user can be encoded into binary format as per the QR code encoding rules following the ISO/IEC 18004 standard. Three encoding modes are set: numeric, alphanumeric and byte mode. The encoding parameters can be set as follows: for numeric—10 bits for every 3 digits, for alphanumeric—a predefined character set with 11 bits for every 2 characters, and for byte mode—encoding each byte as 8 bits. The system can use error correction to read and detect errors in the binary data when generating the QR code. This environment enables the arrangement of the encoded data and error correction codes into the QR code matrix with a basic 21×21 QR code structure.

The bitmap generator can then apply a mask pattern with an alternate black or white base and add format information with error correction level and create a matrix. The format information includes mandatory patterns for finder squares, alignment patterns, and timing patterns as defined by the QR code standard 18004. The system can then convert the matrix into a pixel-based bitmap where each black base becomes a black square and white base in a white square. Then the binary code generator inserts the encoded binary data into the grid using the QR code's placement rule (pre-defined in the system) and ensures that no data overlaps with reserved areas for patterns or error correction. The system renders the QR code as an image for display on the users' device.

To ensure anonymity of the user, the system does not save the user information as it does not create the QR code based on the users' location, IP address or email; but rather the QR code is solely based on users' pseudonym and password. The sign-up QR codes do not attach any metadata (like IP address, timestamps, or geolocation) to the generated QR code or its data. Moreover, the generated one-time QR codes are generated in the same way only with users' device (working as a server) and are not available on the main server or stored on the system. The one-time QR code generation happens entirely on the user's device without server interaction. The system processes all QR code creation steps on the user's device, avoiding server-side processing and it disables IP logging in the app's network stack to ensure no geolocation APIs are triggered during this process.

An example of the system process flow regarding the sign-up QR code includes a bitmap generator accepting users' input of signing up taking a pseudonym and password that is to be encoded. Then it converts the input for QR code creation. The input is converted into binary format using QR code encoding rules mentioned above. The password is encrypted and then masking is done. The information in binary format is converted into picture format as a sign-up QR code and stored on the main server (autonomous server) to be retrieved the next time the user logs in.

One-Time QR Code: The one-time QR codes are also generated following the above process from accepting information from users to generating a one-time QR code except that it all happens on the users' own device ensuring no personal information is stored on the main server.

The user receives a new one-time QR code when they login to the system, yet the users' IP address is never saved on the system. The autonomous servers only process data in the memory, ensuring no persistent records about the user. To generate a one-time encryption key for securely encoding data in the one-time QR code, a cryptographic technique is used with an asymmetric encryption providing public and private key pair. The system encrypts as the encoded data first converted to bytes and then encrypted, by using the recipient's public key. The encrypted data, along with any necessary information is then encoded into the one-time QR code. To decrypt, the one-time key, a private key is used to retrieve the original user data. The system does not store encryption keys in the domain, ensuring secure communication, and designing keys to expire after use to maintain users' privacy and security.

These untraceable one-time QR codes are only visible to user one time with the sign-up QR code so no other entity (robot or person) can access the user profile. Moreover, complete non-traceability does not mean that legal authorities cannot access the system if needed. The information in the one-time QR codes is encrypted with users' sign-up QR code, so that only this system can decode it. This ensures that even if the one-time QR code can be traced, its data remains secure and anonymous.

After an untraceable one-time QR code is generated, the user can be required to complete a two-way authentication (without using phone number or email address). This system introduces a new call and response authentication with a probabilistic Q&A AI method that generates a random question based on user's real-time (stored information) that user needs to answer to login.

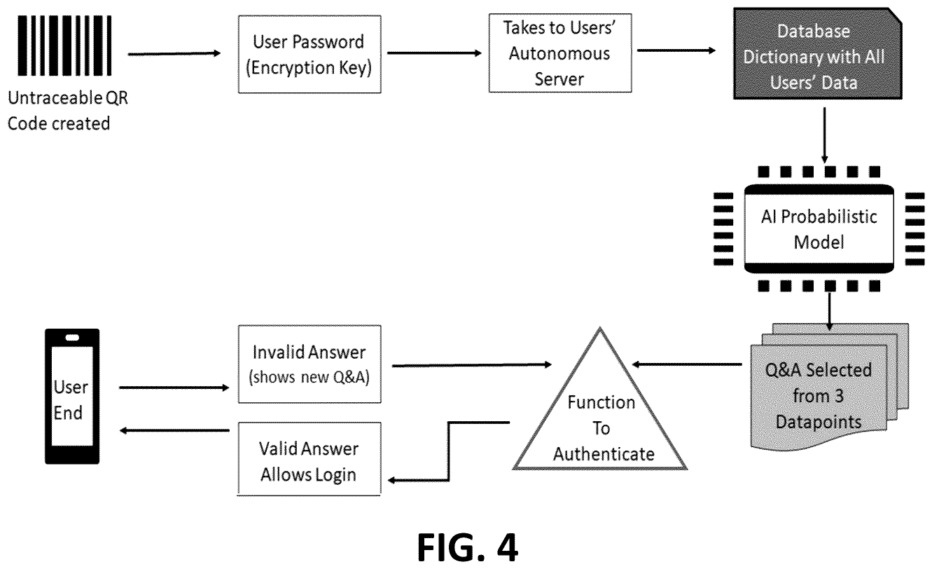

With reference to FIG. 4, shown is an example of a process of call and response authentication. This is done by first using the password as the encryption key to transmit some randomly generated information as the challenge selected on the probability model Q&A, whereupon the user must return the right response with a similarly encrypted value which is some predetermined function of the originally offered information, thus proving that it was able to decrypt the challenge. The challenge is an encrypted question (relevant to user's data) like asking the duration of the user's last workout or GP (general practitioner) visit, while the response is encrypted as 45 minutes (only known by the user), proving that the other end was able to decrypt the randomly assigned challenge. A hash function is applied to a password and a random challenge value to create a response value which uses this AI Q&A probabilistic model to provide randomized challenges and conditioned questions for user authentication without personal information (location). This feature can be switched off upon user's request.

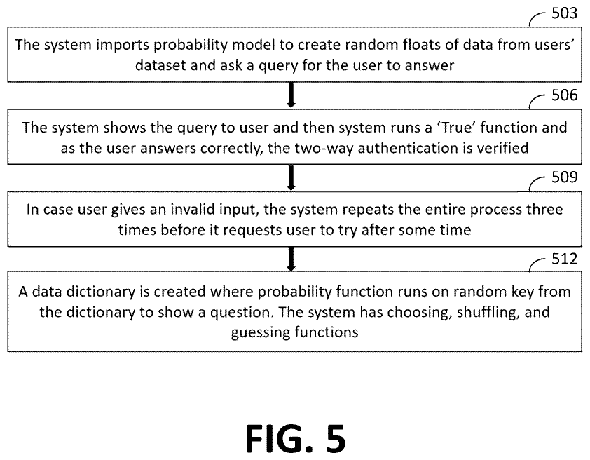

With reference to FIG. 5, shown is an example of a flow diagram illustrating the creation of random Q&A challenges. FIG. 5 illustrates how the AI probabilistic Q&A challenge works. The details of this system for call and response authentication with a probabilistic AI model are presented below.

Initially at 503, a random model can be imported to create an environment, e.g., in Python. This model exists in python libraries and can create random floats of data from the dataset by choosing any data point randomly and then shuffling the data in a dataset to create the probabilistic model. Additionally, a graphical user interface (GUI) toolkit (tkinter) can be imported to support the graphical user interface. The information collected from the user is stored in form of datasets while each type of data goes to its' relevant data directory. When the user clicks on the two-way authentication after the sign-up QR code is created, the app using Python random function randomly selects a data point and creates a query for the user to answer. Each data point along with its' query has been defined and the model has been trained to deliver the new query after the data point randomly selected.

After the user data is recorded in multiple data directories, the app randomly selects a data point—e.g., users' last GP visit and based on this information the system generates a query (e.g., “When you last visited your GP?”) and displays it to the user to answer at 506. The app displays the selected query and when the user inputs the response, it returns the answer to system. The system runs a ‘True’ function and if the user answers correctly, the two-way authentication is verified at 506 and takes user to their home page. If the user gives a wrong answer, the system sees it as an ‘invalid input’ at 509 and repeats the entire process of randomly selecting data point and generating a query again. In case the users' give three wrong answers consecutively at 509, the app locks with an ‘exit’ function and request the user to login after a few minutes again.

In this process of call and response authentication, a dictionary of data points is maintained at the back end with their associated queries at 512. The asking a question function runs on randomly selecting a key from the dictionary and displays the corresponding question. The GUI toolkit (tkinter) function displays the question and returns the result back to app. Because this app uses the new data points for call and response authentication, the system adds new datapoints following these steps. First the data points are added by ‘data set extend’ function. It then updates the datasets and uses defined queries for each data point. Python can be used to implement probabilistic model support choosing, shuffling, and guessing functions in a dataset at 512.

The use of the untraceable sign-up QR codes and one-time QR codes facilitates the secure collection of health data across multiple services. The new data processing operator is added in this system for health data collection from various databases and devices, ensuring devices' verification and processing from multiple datasets using AI simulation model.

This data processing operator receives the datasets sent by the autonomous server from databases that it needs to process. The data can come from various sources, like user input, files, databases, and devices. Then the operator interprets the type of operation it needs to perform with each dataset based on its' type. The operator can have a pre-defined set of functions for each dataset like processing and analyzing blood test reports based on reference values or an AI model to reconstruct coarsely sampled medical images like scans or x-rays and turn them into high-quality images. The operator can perform a specified operation on the datasets as defined in the system. It returns the output result after processing. The operator supports seamless health data gathering from multiple devices and devices verification. Depending on data type, an operator can manipulate information in a specific way to generate a specific result.

The following ways can be followed to authenticate and gather data seamlessly from multiple devices and databases. Personal devices such as the Internet of Things (IoT) of the user can be registered on the system when the user signs up. This can be done by registering the device with a unique identifier issuing a token for each device so the user keeps connected at any time when logging in the system. The non-personal medical devices recording and monitoring health information of the user, like blood pressure monitors, stethoscopes, hearing aids, Holter monitors, x-ray and MRI machines can be authenticated using a mutual authentication method. In this process, the system gives the device (owned by another entity and not the user) a code that the user enters into the system to connect with the device directly or with the database. This ensures that both the device and server authenticate each other. For receiving health data from an Electronic Health Record System (EHR) or Hospital Management Information System (HIMS), third party verification is used. The GP or other medical practitioner authorizes the users' system to receive the medical report or patient's history from EHR directly every time new reports are created.

Cloud storage sync works with operator to receive data from hospitals, medical laboratories. The example of this is: after the X-ray exposure to the patient, the X-ray image displayed on the console of a DDR portable radiography system. With the Wi-Fi connection ability, the X-ray images transferred to the network in DICOM 3.0 standard, which is seen on the users' mobile screen. Creating a system with an AI data operator that uses an AI simulation model to store, process, and analyze information of a user, and then generate results, involves following steps.

The operator can be designed using python. To begin, flask web framework along with various libraries for handling each data type can be installed into this system. Then an operator can be created using flask and python libraries to read and process each dataset type. First library is Pandas which helps in handling and analyzing excel/csv data files. Second python-docx library is used for reading word documents along with NumPy for numerical data handling. Next, to process specialized health images including MRI scan, X-rays, ultrasound, mammograms and CT scans, Pydicom is used. To read these images and making sense of health predicting graphs Matplotlib is downloaded which is an existing Python library. To train AI models and run machine learning algorithms Scikit-learn is used.

The autonomous server segregates different types of datasets and sends to assigned operators. The user opens the app to connect to the autonomous health server where different types of files are uploaded. The server defines how operators receive data and then operators process user data for personalized recommendations and automated decision-making. In the next step data inputs are classified and processed as per their type. The system then generates output results, predictions and recommendations for users'healthcare.

The AI probabilistic Q&A model was trained initially as a model to generate natural language queries based on users' data points. The model used two sets of data types: 1) medical data including lab test reports, prescriptions, general physician's notes, and 2) health data coming from daily activities such as blood pressure, heart rate, step counts, calorie counts and others. Both the data sets were taken together to train the model to generate queries in natural language. The model used a structured format in CSV to store the data points and corresponding queries in the database dictionary of the user.

The pre-defined parameters based on medical reference values were added in the main system that related user generated real-time values, compared it with standard reference values and highlighted any abnormalities. The user generated data points were split into words and numerical and tokens were created (words or sub-words) and a structured representation of numbers generated. Now, the system generates queries and pair the tokenized data points with tokenized queries. This is done through encoder processes where the inputs are the users' data and added as data points, and the decoder generates the query. Each data point is added into the encoder which is part of the data dictionary and then the decoder is trained to generate the query. This training is done by splitting the dataset into training (80% available users' data), validation (10%) and test sets (10% data). The system uses an optimizer with a learning rate scheduler. The system takes the API service for real-time query generation. The system gathers real-time users' data to improve the model's accuracy of generating queries. Every time users generate data, new data points are added as an input and the system trains on a new dataset to evaluate the model's ability to generate accurate and relevant queries. This is only the initial process where the users' generated data is used to analyze the types of data coming into the system and once the training achieves a defined accuracy, the system uses the trained model all times. The system can periodically re-train the model with an expanded users' dataset to enhance generalization.

Combining these data files to perform correlation analysis, an AI simulation model can be used for data processing of each type of data and then an advanced algorithm can be deployed to correlate all reference values for an overall view of users' healthcare. The operator can provide a comprehensive approach to handling multiple types of health data, performs basic analysis, and uses machine learning for correlation and predictions. It expands the meaning of analysis by using the AI simulation model for more sophisticated analyses (e.g., classification or regression models) and includes other techniques like clustering, anomaly detection at a later stage of healthcare data analysis. Data Processing Operator handles filtering, aggregation, sorting, and transformation of data. These operators work on raw data before it's processed by the AI simulation model.

The AI simulation model uses a machine learning method to train and test datasets autonomously and simulate data based on the input features. The proposed operator is responsible for storing, processing, and analyzing user data, as well as applying AI-driven simulations to generate results. The information is then sent to the server to store the results in the system using the relational database of MySQL, creating a structured database. The system also uses cloud storage of large datasets. The AI model used here also pre-trained as part of this system to show information to users based on dynamic sets of data (not just user generated data). This creates an environment which supports one users' data processing effectively with least errors as the AI model has been trained on large datasets. This operator allows the user to interact with the system, to browse and provide feedback, which feeds back into the system for continuous AI model improvement. The operator also helps in basic data processing tasks that the system performs on individual users' information. The task includes removing irrelevant or incomplete data, converting data into a usable format, aggregation and summarizing and joining multiple data sources for a comprehensive analysis. It can give personalized recommendations to user based on a machine learning model to predict users' healthcare trends. The operator can also detect unusual patterns in user data that might help in diagnosis. The operator runs in real-time to efficiently process health datasets.

Reinforcement learning model is used for sequential decision-making that improves over time. The neural networks are designed to understand the complex patterns in data and AI simulation model is also trained on the user collected data. The operator model's performance is analyzed using algorithms and metrics depending on the task. Lastly, cross-validation is performed on datasets to ensure the model generalizes well for future users' data. APIs are implemented to collect and process data in real time and where real-time processing is not needed batch processing is employed to handle large amounts of data. To implement a mechanism to gather user feedback on the system's output, the operator updates the AI models for improve future predictions.

This system uses supervised learning to train the AI model on users' data for, e.g., making health predictions about user. The AI simulation model is built on an AI-based system that uses supervised learning on health datasets to predict health outcomes. First, the system collects users' data and segregates the different types of datasets and then split it into training set for training the model, validation set for tuning and validation, and testing set for final performance of the AI simulation model and its' evaluation (for instance, using 70:20:10 ratio for the training, validation and testing sets respectively). The data trains on a supervised learning algorithm which takes each data point and compares it with its' reference values (based on pre-defined values). This ensures that each data point is either in the normal reference range or not. The system regularly evaluates the model's predictions with real-world data and continuously collects new data to re-training the model.

An example case includes the prediction of diabetes risk by taking users' weight, exercise routine, sugar intake and family history data points. The user is overweight with a BMI of 28, have no exercise routine exercise hours are 0, sugar intake is 80 grams each day and a family history of diabetes. The AI simulation model feeds this information into the system where each data point is compared with its' reference value (i.e. BMI=<26, exercise hours <=3 hrs., sugar intake <=40 grams, and history of diabetes=Yes), and predicts the higher risk of diabetes in the user.

The operator also regularly updates the system with new features, enhanced AI models, and improved data operators, monitor for errors, optimize for speed, and address any system bottlenecks. Building a system with an AI data operator involves several layers: defining system requirements, designing architecture, developing data processing operators, integrating AI models for simulations, and maintaining the system for continuous improvement. With the right combination of AI models, data processing operators, and feedback loops, the system can store, process, analyze, and generate insightful results based on user data. This approach can be applied in various domains, such as personalized recommendations, predictive analytics, and dynamic decision-making systems.

The system based on individual user needs, integrates multiple types of datasets including excel files, word documents, images like X-rays and MRIs, ECG reports, and data from a mobile health app and performs data analysis by correlating information from all these different sources.

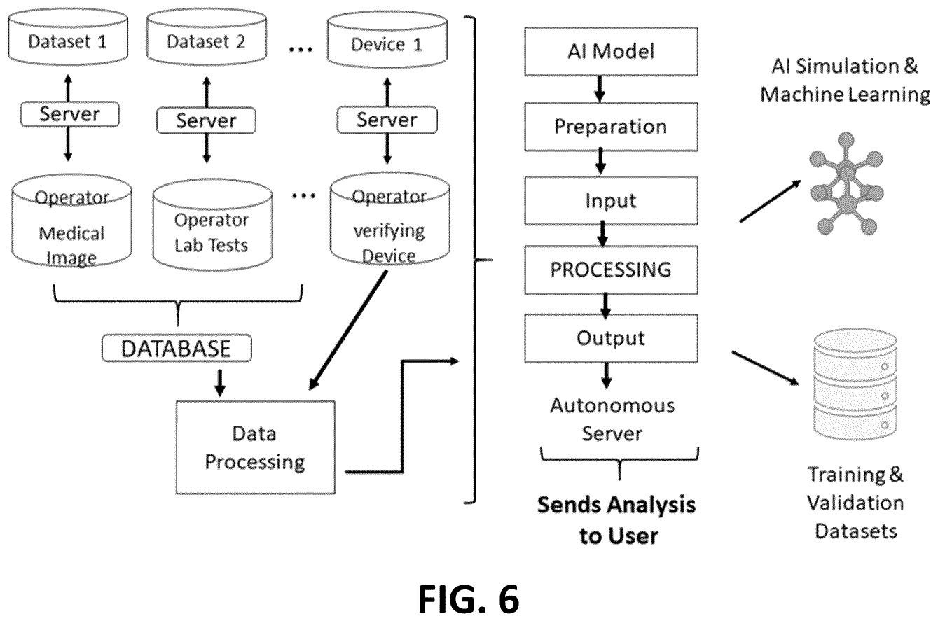

With reference to FIG. 6, shown is an example of data transfer, device verification, and data processing. FIG. 6 illustrates the process for data collection from various datasets, detailing the flow of data from multiple sources into a central data processing unit for processing and storage.

As shown in FIG. 6, different datasets and devices link to the server, where these are classified and send information to the operator. Each have their own assigned servers connecting to their operators to interact with the system. These operators are not only sending data to the central system but also receiving information from the user. Once the data is sent from the servers, it is directed to a central data processing system. This system follows a well-defined data process flow comprising collecting, preparing, input, processing, and output.

Following the five-step data process flow, the central system leverages advanced ML and AI models to further analyze and extract insights from the data. These models can be designed to process vast amounts of data efficiently, recognizing patterns, making predictions, and delivering valuable insights to the organizations. After the data is processed by the AI simulation model, it can be stored either on cloud-based servers or local servers, depending on the preferences and needs of the users.

FIG. 6 illustrates the diverse range of databases that can be used in this system. These databases include, but are not limited to, health data from hospitals, insurance companies, pharmacies, IoT and health apps. The system can be designed to accommodate the varying requirements of these devices and databases, ensuring that data is processed in a way that aligns with their operational goals and regulatory standards.

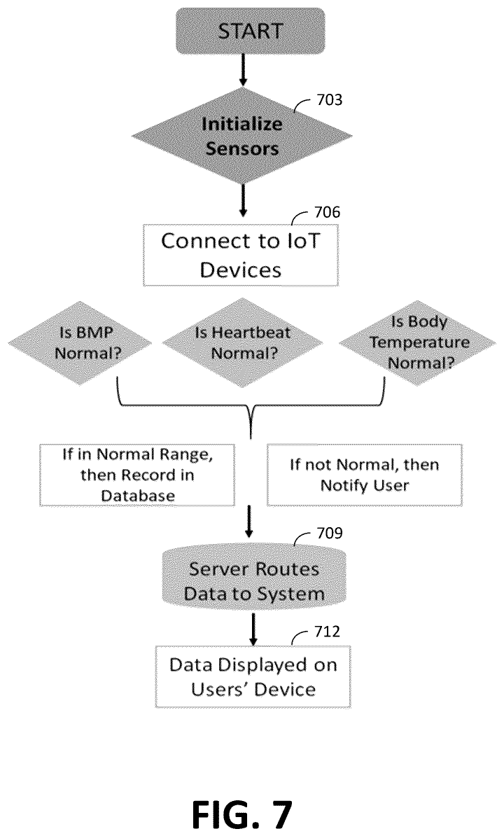

With reference to FIG. 7, shown is an example of drawing of seamless user health data gathering and storing. FIG. 7 illustrates a flow diagram depicting the seamless health data gathering and storing process, which is designed to efficiently capture and manage health information from various sources. Beginning at 703, sensors are initialized to connect to Internet of Things (IoT) devices at 706. It is designed for data collection from multiple inputs, such as wearable devices, mobile health apps, health watch and apps (like Fitbit). Each data source can be connected to a central processing unit, where the incoming data can be validated to ensure accuracy and completeness. If the data is valid, it flows into a secure storage system, where it is organized and stored for future access and analysis at 709. In case of any discrepancies or missing information, the system prompts an error handling mechanism that may involve notifying the user or requesting additional data. Furthermore, the flow diagram shows that data such as heartbeat, BMP and temperature are recorded if normal or else user is being notified about the abnormal value. This incorporates a feedback loop, allowing users to review and confirm the collected data, thus ensuring that the stored health information remains reliable and up to date. This flow diagram effectively visualizes the streamlined process of gathering and storing health data in this system.

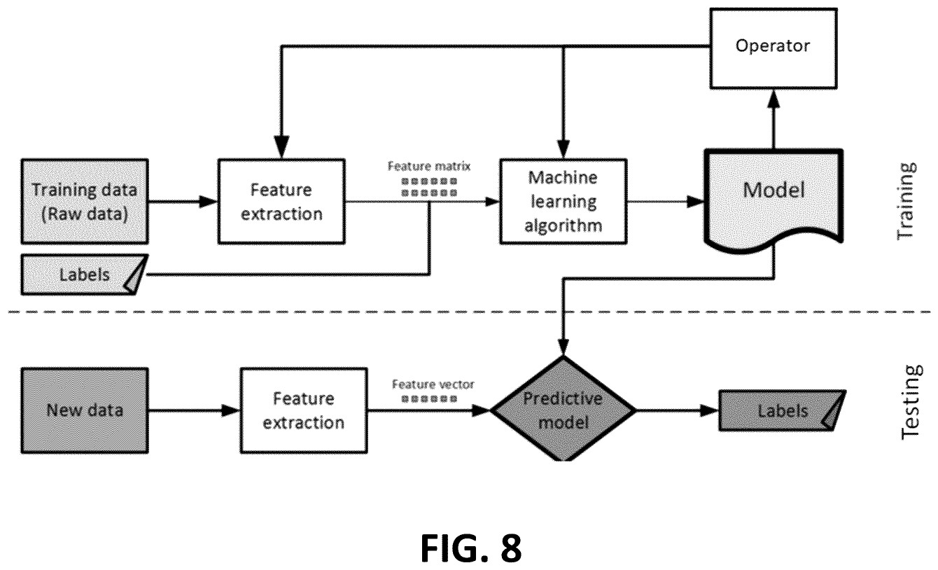

With reference to FIG. 8, shown is an AI simulation model for data processing and recording new data according to various embodiments. FIG. 8 shows the flow of data processing and recording new data using an Artificial Intelligence (AI) simulation model. The model is divided into two main sections: training and testing. In the training section, raw data is first collected and preprocessed, where it is cleaned and transformed to ensure it is suitable for training the model. This data is then fed into a machine learning algorithm, which learns patterns and relationships within the data by adjusting its internal parameters. The training process continues iteratively until the model achieves an optimal level of accuracy. Once the model is trained, it is tested to validate its performance on unseen data, fine-tuning the model as needed.

In the testing section, the trained model is applied to new, unseen data. Here, the model uses the learned patterns from the training phase to test the model and generate insights. The autonomous server can seamlessly collect real-time health data from users and autonomously manage the entire machine learning pipeline without human intervention. This system can collect a diverse range of health datasets and its' training model can learn to recognize a broader spectrum of patterns, variations, and anomalies across different populations, conditions, and physiological profiles. This diversity improves the model's ability to generalize, reducing bias and increasing its effectiveness in accurately predicting health outcomes for individuals with varying genes and medical conditions. Thus, the system can continuously update and fine-tune its predictive models as new data comes in, enabling adaptive learning personalized to individual users. As this system will accumulate more diverse and temporally rich datasets, it will identify subtle patterns and correlations across physiological signals, lifestyle factors, and historical health outcomes. This iterative self-optimization process enhances the model's generalization capability, resulting in steadily improving accuracy in predicting a wide range of health conditions, from predicting chronic disease onset to acute health risks. This flow enables the system to continuously learn from data and provide accurate predictions when presented with new input. The seamless integration of training and testing ensures the model evolves with new data, improving its predictions over time.

The encryption layer protects sensitive information before it is stored or processed by AI algorithms. The encryption process ensures confidentiality using encryption algorithms including AES (Advanced Encryption Standard) is used depending on the sensitivity of the data. Then server's illustration is given showcasing AI algorithms to process data. AI plays a crucial role in analyzing the data, extracting insights, and performing specific tasks while generating user centered reports.

Next, datasets in main database are given. After encryption and AI processing, the processed data is stored in a centralized web-based database. This database is structured using MySQL. The database can be optimized for fast access and retrieval of data, ensuring the system can scale efficiently to handle large volumes of data, especially from government or organizational users. Once the data is stored, it can be made accessible via web servers. These servers can communicate with the database and serve the processed data to the frontend using different programming languages including Python, PHP, HTML. HTML is used to structure the web pages that display the data on the frontend. It can define the layout, elements, and overall content presentation. PHP (Hypertext Preprocessor) can be used on the server side to handle backend logic, such as database queries, form submissions, or session handling. It can connect with the database to fetch processed data and render it dynamically on the web pages. Python can be used for AI processing and data manipulation on the backend. It also can interact with the database and generate data insights or reports.

Finally, the processed data can be displayed on the frontend of the system. The frontend is the part of the system that users interact with directly. It can be designed to be user-friendly, responsive, and capable of presenting complex datasets in an understandable format.

Health data can be stored with privacy and security measures and AI models can be used to create an AI health representation for each user. This systems' output can help the user to have a personal Digital Health Object (DHO). A digital health object is a virtual representation of a health-related physical object, system, or process that continuously updates to reflect real-world changes. It can be used for monitoring, simulation, analysis, and decision-making.

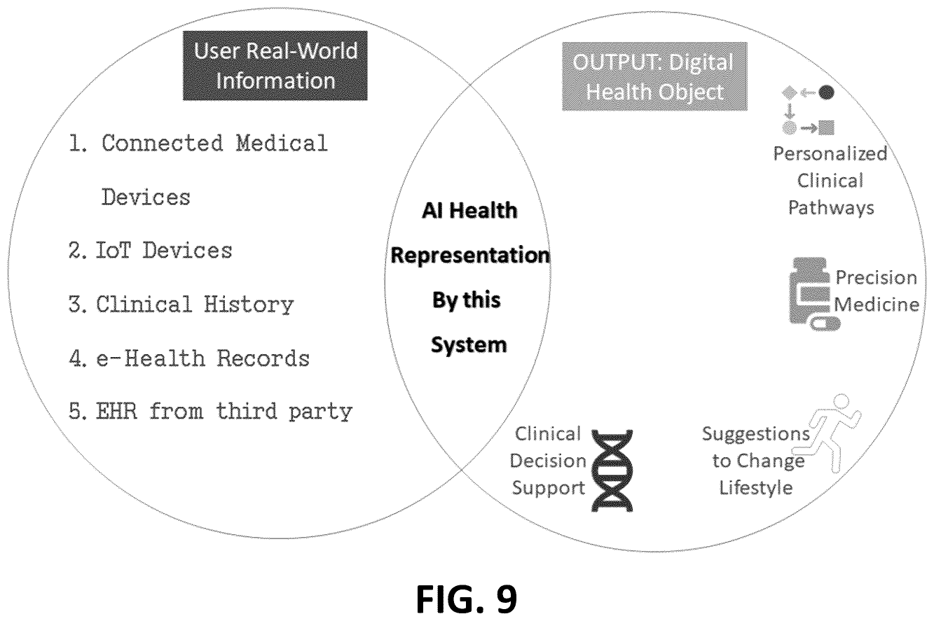

With reference to FIG. 9, shown is an example of a user specific digital health object and its use in healthcare. FIG. 9 draws on how real-world information about a user is collected to create a user specific digital health object. This DHO can support in virtual healthcare based on the users' personalized information. The digital health object is created as a software-based virtual healthcare model of the user. This object can be updated in near real-time to mirror the conditions of the physical system. Users interact with the DHO using this system and connecting with the AI simulation operator. Creating a digital health object of the user can have various benefits like prevention of chronic illnesses, timely diagnosis, treatment decisions, testing treatments in a virtual environment before giving treatment to actual patients and even administration of precision medicine as per the needs of each individual user.

This AI health representation provides the real-time status of the physical systems connected with the main server, testing scenarios without affecting the physical system. The AI based digital health object assigned to each user optimizes and improves efficiency and performance of the overall healthcare system. The user-specific digital health object and provides insights into the users' healthcare data.

The DHO can help in diagnosing health related problems before they become critical, use historical and real-time data to predict healthcare anomalies and guides users for maintaining a healthy lifestyle.

The AI simulation operator can support implementation of a Machine Learning (ML) model to analyze complex algorithms, patterns, make predictions, and provide insights based on the incoming data. It can mimic a digital environment where the physical environment of user is the basis for such simulation. These derived environments from the digital health object can be applied to the physical system through automated decision making. Digital health objects bridge the physical and digital worlds, enabling smarter decisions, reduced costs, and better performance across industries. The automated digital health object adjusts its' data processing and operations using feedback loops with the user. Finally, the AI digital health representations or personalized digital health objects can be exploited for patient treatment planning.

The digital health data bank, which comprises an ordered listing of executable instructions for implementing logical functions, can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can contain, store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory. In addition, the scope of the certain embodiments of the present disclosure includes embodying the functionality of the preferred embodiments of the present disclosure in logic embodied in hardware or software-configured mediums.

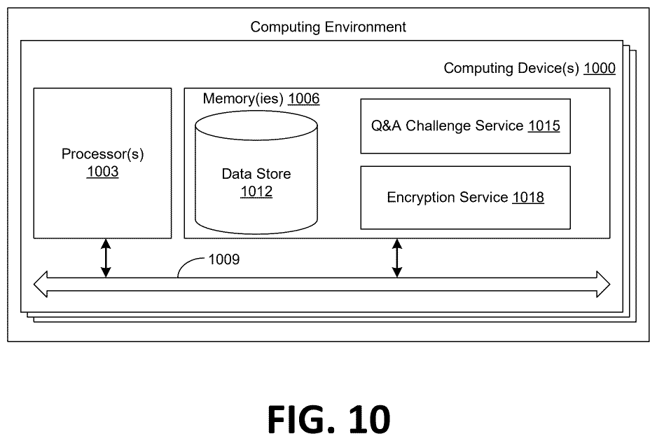

With reference to FIG. 10, shown is a schematic block diagram of the computing environment in accordance with various embodiments of the present disclosure. The computing environment includes one or more computing devices 1000. Each computing device 1000 includes at least one processor circuit, for example, having a processor 1003 and a memory 1006, both of which are coupled to a local interface 1009. To this end, each computing device 1000 may comprise, for example, at least one server computer or like device. The local interface 1009 may comprise, for example, a data bus with an accompanying address/control bus or other bus structure as can be appreciated.

Stored in the memory 1006 are both data and several components that are executable by the processor 1003. In particular, stored in the memory 1006 and executable by the processor 1003 is a Q&A challenge service 1015, an encryption service 1018, and potentially other applications. Also stored in the memory 1006 may be a data store 1012 and other data. In addition, an operating system may be stored in the memory 1006 and executable by the processor 1003.

It is understood that there may be other applications that are stored in the memory 1006 and are executable by the processor 1003 as can be appreciated. Where any component discussed herein is implemented in the form of software, any one of a number of programming languages may be employed such as, for example, C, C++, C #, Objective C, Java®, JavaScript®, Perl, PHP, Visual Basic®, Python®, Ruby, Flash®, or other programming languages. Additionally, it is understood that terms such as “application,” “service,” “system,” “engine,” “module,” and so on may be interchangeable and are not intended to be limiting.

A number of software components are stored in the memory 1006 and are executable by the processor 1003. In this respect, the term “executable” means a program file that is in a form that can ultimately be run by the processor 1003. Examples of executable programs may be, for example, a compiled program that can be translated into machine code in a format that can be loaded into a random access portion of the memory 1006 and run by the processor 1003, source code that may be expressed in proper format such as object code that is capable of being loaded into a random access portion of the memory 1006 and executed by the processor 1003, or source code that may be interpreted by another executable program to generate instructions in a random access portion of the memory 1006 to be executed by the processor 1003, etc. An executable program may be stored in any portion or component of the memory 1006 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1006 is defined herein as including both volatile and nonvolatile memory and data storage components. Volatile components are those that do not retain data values upon loss of power. Nonvolatile components are those that retain data upon a loss of power. Thus, the memory 1006 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1003 may represent multiple processors 1003 and/or multiple processor cores and the memory 1006 may represent multiple memories 1006 that operate in parallel processing circuits, respectively. In such a case, the local interface 1009 may be an appropriate network that facilitates communication between any two of the multiple processors 1003, between any processor 1003 and any of the memories 1006, or between any two of the memories 1006, etc. The local interface 1009 may comprise additional systems designed to coordinate this communication, including, for example, performing load balancing. The processor 1003 may be of electrical or of some other available construction.

Although the response Q&A challenge service 1015, the encryption service 1018, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flow diagrams of FIGS. 4-8 show examples of the functionality and operation of implementations of components described herein. The components described herein can be embodied in hardware, software, or a combination of hardware and software. If embodied in software, each element can represent a module of code or a portion of code that includes program instructions to implement the specified logical function(s). The program instructions can be embodied in the form of, for example, source code that includes human-readable statements written in a programming language or machine code that includes machine instructions recognizable by a suitable execution system, such as a processor in a computer system or other system. If embodied in hardware, each element can represent a circuit or a number of interconnected circuits that implement the specified logical function(s).

Although the flowcharts and sequence diagram show a specific order of execution, it is understood that the order of execution can differ from that which is shown. For example, the order of execution of two or more elements can be switched relative to the order shown. Also, two or more elements shown in succession can be executed concurrently or with partial concurrence. Further, in some examples, one or more of the elements shown in the flowcharts can be skipped or omitted.

Also, one or more or more of the components described herein that include software or program instructions can be embodied in any non-transitory computer-readable medium for use by or in connection with an instruction execution system such as, a processor in a computer system or other system. The computer-readable medium can contain, store, and/or maintain the software or program instructions for use by or in connection with the instruction execution system.

A computer-readable medium can include a physical media, such as, magnetic, optical, semiconductor, and/or other suitable media. Examples of a suitable computer-readable media include, but are not limited to, solid-state drives, magnetic drives, or flash memory. Further, any logic or component described herein can be implemented and structured in a variety of ways. For example, one or more components described can be implemented as modules or components of a single application. Further, one or more components described herein can be executed in one computing device or by using multiple computing devices.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A method for secure data transfer to a digital health data bank, comprising:
login, via a user device, to an autonomous server of the digital health data bank, the login comprising an authentication based upon a sign-up QR code associated with a user of the autonomous server and a random question & answer (Q&A) challenge; generating, by the user device, a one-time QR code comprising user data based upon the sign-up QR code; and
transmitting, by the user device, the one-time QR code to the autonomous server.

2. The method of claim 1, wherein the sign-up QR code is generated during initial set up of the autonomous server by the user.

3. The method of claim 2, wherein the sign-up QR code is a unique identifier that is not directly linked to personally identifiable information of the user.

4. The method of claim 2, wherein the sign-up QR code is generated based upon input of the user.

5. The method of claim 1, wherein the login to the autonomous server comprises:
providing, by the user device, the sign-up QR code and user password to the autonomous server;
receiving, by the user device, an encrypted question from the autonomous server, the encrypted question comprising a randomly generated question based upon user specific information of the digital health data bank; and
providing, via the user device, an encrypted response to the randomly generated question.

6. The method of claim 5, wherein the encrypted question and encrypted response are encrypted based upon the user password.

7. The method of claim 6, wherein login validation is based at least in part upon encryption of the encrypted response.

8. The method of claim 5, wherein the randomly generated question is provided by an AI probabilistic model based upon the user specific information.

9. The method of claim 5, wherein the randomly generated question is provided based upon a random key of a data dictionary associated with the user.

10. The method of claim 9, wherein the random key is selected based upon a choosing, shuffling or guessing function.

11. The method of claim 5, wherein validation of the login is within a defined number of attempts.

12. The method of claim 5, further comprising:
receiving, by the user device, a second encrypted question from the autonomous server, the second encrypted question comprising a second randomly generated question based upon the user specific information; and
providing, via the user device, a second encrypted response to the second randomly generated question.

13. The method of claim 1, wherein generating the one-time QR code comprises:
encrypting the user data using a public key of a recipient; and
generate the one-time QR code with the encrypted user data using the sign-up QR code.

14. The method of claim 13, wherein the user data is stored in the digital health data bank based upon the sign-up QR code and decrypted based upon a private key of the recipient.

15. The method of claim 13, wherein the user data is stored in a physical server or a cloud server.

16. The method of claim 1, comprising obtaining the user data from a database containing healthcare information associated with the user.

17. The method of claim 1, wherein the user data comprises a medical report, prescription, insurance claim, medical scan, or Internet of Things data.
